(19) United States
(12) Patent Application Publication
(10) Pub. No.: US 2010/0203104 A1
(43) Pub. Date: Aug. 12, 2010

(54) DELIVERY SYSTEM FOR RISPERIDONE

(75) Inventors: Wouter De Graaff, Oss (NL); Armin Szegedi, New York, NY (US)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. Organon

(21) Appl. No.: 12/515,882

(22) PCT Filed: Nov. 21, 2007
(86) PCT No.: PCT/EP2007/062628

§ 371 Date: Jan. 28, 2010

(30) Foreign Application Priority Data
Nov. 22, 2006 (EP) 06124598.1

(51) Int. Cl. A61K 9/00; A61K 31/519; A61P 25/18

(52) U.S. Cl. 424/432; 514/259.41; 424/433

(57) ABSTRACT

This invention relates to an extended release formulation comprising solid risperidone, which formulation is a vaginal device having a skin and which device comprises an inner compartment made of a thermoplastic polymer, which polymer is containing risperidone. The polymer is preferably made of ethylene-vinyl acetate copolymer.

The present invention relates to an extended release formulation comprising solid risperidone and to a method of manufacture.

Risperdal® (risperidone) is an atypical antipsychotic agent and is widely used in psychiatry for the symptomatic management of psychotic disorders including bipolar mania and schizophrenia. Risperidone belongs to the chemical class of the benzisoxazole derivatives and the chemical designation is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one. U.S. Pat. No. 4,804,663 describes a synthesis of risperidone.

For psychotic disorders chronic use and medication compliance is necessary for therapeutic results. Medication compliance is a complex and important subject, since insufficient compliance causes reduced personal prospects for patients and also considerable financial costs. All kinds of patients experience difficulties in staying adherent to medication, including the old and mentally infirm. The reasons for non-compliance are varied and include forgetfulness, over-complex dosing schedules, occurrence of side-effects by peaks in fluctuations of plasma levels of the drug and inconvenient administration of the drug by pain at the injection site. In particular schizophrenic patients have heightened problems to adhere to medication and medical practitioners are concerned about compliance in order to improve their patients' well being.

Long-acting risperidone (Risperdal Consta®) is an aqueous suspension of microspheres comprising risperidone and a biodegradable copolymer for intramuscular administration with incidence of injection site pain. The method for preparing the microspheres having a substantial sigmoidal release profile with an initial lag phase is described in U.S. Pat. No. 6,596,316. The typical starting dose of Risperdal Consta® is 25 mg every 2 weeks. Depending on an individual's response, the dose can be increased to a maximum of 50 mg every 2 weeks. Currently, Risperdal Consta® is available in strengths of 12.5 mg, 25 mg, 37.5 mg and 50 mg injections. The product causes less plasma drug fluctuation than the oral formulation. Significant release of risperidone from the microspheres begins 3 weeks after the first injection, thus administration of an oral antipsychotic is necessary during this period. Steady-state plasma concentrations are reached after the fourth injection. Effects of long-acting risperidone persist for at least two weeks after injection, the time period needed for degradation of the microspheres comprising the risperidone. Obviously, medication of the patients cannot be interrupted in this period of two weeks in case of side-effects. Elimination is complete 7-8 weeks after the last injection (Harrison, T. S., and Goa, K. L. Long-acting risperidone: review of its use in schizophrenia. CNS Drugs (2004), 18:113-132).

US 2003/0153983 describes implantable medical devices that provide resistance to microbial growth on and in the environment of the device and resistance to microbial adhesion on the device. In U.S. Pat. No. 4,469,671 a contraceptive device for intravaginal use is described comprising a bioinsoluble, biocompatible polyurethane and an acrosin inhibitor such as salts of alkyl or alkenyl sulfate. An elastomeric vaginal ring comprising a pharmacologically active compound or pharmaceutically acceptable addition salts for the treatment of cancer is described in U.S. Pat. No. 5,558,877. An elastomeric matrix type of system for vaginal delivery of antimicrobial agents is described in WO 02/076426. U.S. Pat. No. 4,016,251 discloses a drug-delivery device comprising of a shaped body of ethylene-vinyl acetate containing a drug and permeable to passage of the drug by diffusion.

In the context of psychiatric drug treatment it is highly unusual to contemplate a vaginal delivery system for extended release, although the intravaginal route of administration was mentioned before in a broad list of possibilities for administering antipsychotics (WO 2004041118). WO 03/055424 and WO 2005/004837 describe selected serotonin reuptake inhibitors for use in an extended release formulation in the form of a vaginal delivery system.

Rather, the vaginal route of administration appears acceptable for contraceptive regimes or hormone replacement therapies which are exclusively aimed at treatment of the female person. In general, vaginal delivery devices are well-known in the field of gynaecology for the delivery of hydrophobic steroidal drugs for contraceptive uses, such as exemplified in U.S. Pat. No. 4,292,965, WO97/02015, WO2004/103336, WO2005/089723 and EP 0 876 815. A contraceptive vaginal ring is marketed under the trademark Nuvaring® by Organon, the Netherlands. Such rings are designed for the purpose of administering high potency steroids, for which drug delivery rates in the order of 0.01 to 0.5 mg/day are usually sufficient to obtain beneficial therapeutic effects. However, for risperidone therapeutically effective amounts to be delivered locally is much higher and ranges in the order of approximately 0.5 to 10 milligrams a day. For improving patient compliance side-effects as a result of peaks in plasma levels are to be minimized and therefore the delivery of risperidone from rings is bound to controlled rates and to low burst release.

Extended release devices in the form of a vaginal delivery system for selected serotonin reuptake inhibitors as described in WO 03/055424 include, as drug containing compartment, one or more channels in the surface or a pocket molded in the ring or a hollow toroid polydimethylsiloxane tubing for use. WO 2005/004837 describes a device with a reservoir containing dispersed active agent and a sheath discontinuously surrounding the reservoir. WO0170154 discloses a siloxane elastomer vaginal ring device with a bore located in the ring comprising an oxybutynin composition, wherein the bore runs from the surface of the ring into the ring. For non-steroidal drugs the choice for polysiloxane polymers relates to their high drug solubility and the well known high permeability of polysiloxane polymers (A. D. Woolfson, R. K. Malcolm, R. J. Gallagher, Journal of Controlled Release 91 (2003) 465-476). In addition, the diffusion coefficient for the same type of molecules in polysiloxanes is typically 100 to 200 times higher than the diffusion coefficient found in polyvinyl acetate copolymers (poly-EVA) (Treatise on controlled drug delivery; fundamentals, optimization, applications, edited by A. Kydonieus, Marcel Dekker Inc. New York, 1992. Typical diffusion coefficient for steroids, pp. 66-67).

Unexpectedly, it has now been found that an extended release formulation in the form of a vaginal delivery system can be prepared for risperidone with superior drug delivery characteristics in terms of a release almost lacking initial burst, of a substantially constant high release rate in the range of approximately 0.5 to 10 mg/day for a period of more than one week up to one or two months, in combination with a high efficiency in delivered risperidone, and which system has optimal mechanical properties, in particular flexibility, by avoiding the use of polysiloxane as taught in the prior art.

The present invention provides for a vaginal device comprising solid risperidone, a skin and an inner compartment, which inner compartment is made of a thermoplastic polymer, which polymer is containing solid risperidone. Preferably, the skin is a substantially continuous cover over the inner compartment. Good results can be obtained when the inner compartment contains 5-80 wt % of risperidone. Preferably, the inner compartment comprises a core, which does not contain solid risperidone. Preferably, the inner compartment, and/or the skin, and/or the core or all three of these is or are made of ethylene-vinyl acetate copolymer. In a more specific embodiment an ethylene-vinyl acetate copolymer having a vinyl acetate content in the range of 6 to 40% is used.

An extended release formulation according to the invention comprising risperidone has the advantage of non-invasive administration, of providing drug release immediately upon exposure of the formulation to aqueous media, of immediate interruption of drug delivery after removal of the system from the vagina, which is particularly advantageous in case medical practitioners have the incentive to interrupt or change the treatment for reasons related to insufficient therapeutic effect or to serious adverse effects during treatment. In addition, the formulation according to the invention advantageously delivers risperidone without significant initial burst through the entire surface area of the device and therefore minimizes the risk of high risperidone concentrations at the vaginal tissue. Moreover, the extended release formulation in the form of a device according to the invention improves compliance with drug treatment in view of the ease with which the formulation can be applied and removed by the women in need of treatment.

Advantageous characteristics of the invention are that the device can easily be manufactured using extrusion techniques and is flexible in view of the small cross-sectional diameter if manufactured in the form of a ring. In addition to that, the extended release formulation according to the invention has an intrinsically safe design against dose-dumping. By application of a core in the inner compartment, the system allows for an improved drug substance efficiency. Application of a core also allows to tune mechanical properties of the system which are relevant in relation to comfort (foreign body feeling) and retention without affecting release kinetics significantly.

The presence of risperidone in solid form provides for a sufficient and continuous supply of risperidone during release and the solid form prevents crystallisation of the drug on the outside of the device during manufacturing.

### Clarification of Terminology.

With a vaginal device a drug delivery system for insertion into the vagina of a woman is meant. The system has preferably the form of a ring, such that the delivery system has an elongated shape of which the two ends are joined together. The ring may comprise one or more loops and those loops may have various shapes, such as oval, ellipsoidal, toroidal, triangular, square, hexagonal, octagonal, etc. Alternatively, the system according to the invention is helically-shaped, which means the shape of a fibre helix with more than one loop and two ends which are not joined together.

With risperidone is meant 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one. Risperidone is a non-ionized drug, having a molecular weight below 500 Dalton and having a solubility of 0.1 wt % in ethylene vinyl acetate copolymer having a vinyl acetate content of 28%. Solubility is measured as described in Laarhoven, J. A. H., et al. (2002), International Journal of Pharmaceutics 232, page 165. The salts of risperidone are unsuitable for use in the formulation according to the invention.

The solid form required to obtain the high load of 5 to 80 wt % of risperidone in the polymer is preferably crystalline risperidone. The crystals will effectively be dispersed within the polymer of the inner compartment. Another reason to require the presence of solid risperidone is to obtain the extended delivery of risperidone from within the inner compartment as will be explained in more detail herein below.

With continuous skin is meant that the skin is continuously surrounding the risperidone containing compartment and is devoid of expressly provided parts in the skin for release of the drug. Thus, direct contact between vaginal tissue and drug compartment is minimised in order to avoid local irritation. The skin is substantially continuous in the sense that only incidental apertures may be present for example, the ends of a helically shaped system or apertures due to shear during manufacturing or due to incomplete closure of ring ends, but such openings are not purposefully introduced into the skin in order to facilitate the passage of risperidone through the skin. It is not excluded that the skin material may comprise dissolved risperidone.

An inner compartment is the compartment which contains the risperidone to be delivered to the patient and is covered by the skin. Therefore, there is no direct contact between the vaginal tissue and the inner compartment. The skin is the barrier protecting the vaginal tissue from undesirable local effects from the concentrated drug in the inner compartment. The inner compartment is formed by a thermoplastic polymer.

A core is an inner structure within the inner compartment and serves to reduce the drug containing space in the inner compartment. The core does not contain solid risperidone. It is not excluded, though, that the core material may comprise dissolved risperidone.

When risperidone is loaded into the inner compartment during the production process some risperidone may enter into the core. The core can be made of any suitable material such as a polymer or the thermoplastic polymer used for the inner compartment. The core can also contribute to the strength or flexibility of the device and to increase drug substance efficiency. In another context the inner compartment is also referred to as an intermediate layer when a core is present in the device.

The delivery system according to the present invention shows superior drug delivery characteristics in terms of a substantially constant high release rate during its duration of use and almost lacking initial burst release. The reduced burst release is defined by the burst-factor, which is defined as

$$\frac{D 1 - D 2}{D 2}$$

with D1 and D2 the in vitro release rate measured on day 1 and day 2 respectively. For the present invention the burst-factor is in the range of −1.0 to +3.0, preferably in the range of −0.5 to +1.5 and most preferably in the range of −0.5 to +0.5.

The substantially constant release is defined by the constant release factor, which is calculated by dividing the average in vitro release rate of the first half of the release curve (AVG1) in mg/day excluding day 1, by the average release rate of the second half of the release curve (AVG2) in mg/day (e.g. for an in vitro release curve measured for 24 days the average release from day 2 to day 12 is divided by the average release from day 14 to day 24). For the present invention the constant release factor is in the range of 0.80 to 1.20 and preferably in the range of 0.85 to 1.15.

The present invention provides for delivery rates of risperidone in the range of 0.5 to 10 mg/day for a period of use of from one week up to 1 or 2 months.

The characteristic of the invention may be understood and influenced by the following explanation and use thereof: Fick's law of diffusion governs the release of compounds. Vaginal rings are cylindrical reservoir/membrane designs of which the release rate can be described by the equation below. Suitable rings can therefore be made by an appropriate choice of the parameters that affect the release rate.

The release rate of a cylindrical reservoir/membrane design is:

$$\frac{d M}{d t} = \frac{2 π {LD}_{p} {K}_{p / s} Δ C}{Ln ( {r}_{0} / {r}_{1} )}$$

L the length of the cylinder
Dₚ the diffusion co-efficient of the compound in a skin polymer
K_{p/s}=partition coefficient of the compound between the skin and inner compartment
ΔC=the difference in concentration of dissolved risperidone between the inner compartment near the skin and the sink
r₀=is the overall radius, i.e. the cross-sectional diameter including the skin
rᵢ=is the radius of the inner compartment (i.e. r₂/r₁=1) or of the core

The equation shows that zero order release is obtained when the term on the right-hand side of the equation is constant, i.e. not a function of time.

It is shown in FIGS. 2 to 4 that substantially constant release rates of risperidone of approximately 0.5 to 10 mg/day can be achieved with the devices according to the invention having a skin substantially continuously covering the inner compartment. Apparently, the solubility of risperidone in ethylene-vinyl acetate (EVA) of the inner compartment is such that the ΔC for risperidone is high enough to provide for fast release kinetics. The limiting factor in maintaining a substantially constant ΔC in a quasi steady state with a high release rate of risperidone, i.e. maintaining a substantially constant drug delivery from the device in the presence of a relatively thin skin with low barrier properties, is the supply of dissolved risperidone to the interface between the inner compartment and the skin. The supply (or referred to as release rate) is the result of a complex mass transport process determined by factors including the dissolution rate of risperidone into the polymer, which in turn is determined by the solubility of risperidone in the polymer and the surface area of the drug exposed to the polymer. The latter is determined by particle size, shape and drug content. Also the diffusion rate of risperidone through the polymer is an important factor for the dissolution and release rate. It has been found that devices having risperidone in the inner compartment in the range of 30 to 70 wt % not only provide for fast release rates but also provide substantially constant release kinetics.

It is believed that with risperidone contents in the polymer above approximately 30 wt. % drug particles can be close to each other within the polymer of the inner compartment. The structure formed by the dispersed solid particles in the polymer depends on drug content and additionally on particle size and shape. During drug release, the properties of the inner compartment itself change in time by the slow dissolution of the drug particles, apparently facilitating drug dissolution and transport rate resulting substantially constant high release rates. Probably the formation of improved diffusion pathways in the polymer by the progressively dissolving particles leaving voids in the polymer and the simultaneous flow of aqueous liquids through the skin into the inner compartment filling the voids with water are important factors in achieving substantially constant release at high levels of drug content.

In the delivery devices of the invention risperidone is present in all polymer layers. When a drug in the manufacturing process of the system is loaded into the inner compartment, the drug diffuses during the production process and/or during storage of the system to the other polymer layer(s) up to equilibrium concentration.

In line with the concept of the core comprising ring, for a ring without core the lengthening of the diffusion distance should also be kept as small as possible and the active compound should also be present in the solid form in order to obtain essentially zero-order release kinetics. Lengthening of the diffusion distance in case of the ring without core can be kept relatively small by keeping the cross-sectional diameter of the inner compartment relatively small. Such a small diameter also results in a relatively small volume of the inner compartment and hence, the amount of active compound, which is required to sustain the release for the intended period of use, is loaded in high concentration in the inner compartment.

A high concentration of active compound in the inner compartment of a ring without core also could be achieved in a large diameter ring, but this would require the use of a large excess of active compound, i.e. much more than required to sustain the release over the intended period of use and hence, this results in an economically and environmentally less attractive dose form with a low drug substance efficiency.

In analogy with a small inner compartment volume of the ring without core, a small inner compartment volume of the core comprising ring serves the purpose of concentrating the active compound in a relatively small polymer volume during processing.

The vaginal delivery system according to the present invention can provide a release rate of risperidone in the range of 0.5 to 10 mg/day for a period of use of from one week up to 1 or 2 months. Preferably the rate is in the range of 1.5 to 5 mg/day.

The thermoplastic polymer that can be used in making the drug delivery system according to the present invention may in principle be any extrudable thermoplastic polymer material suitable for pharmaceutical use, such as ethylene-vinyl acetate (EVA) copolymers, low density polyethylene, polyurethanes, and styrene-butadiene copolymers. In a preferred embodiment, ethylene-vinyl acetate copolymer is used due to its excellent mechanical and physical properties. The EVA copolymer may be used for the core, the intermediate compartment (inner compartment) as well as the skin and can be any commercially available ethylene-vinyl acetate copolymer, such as the products available under the trade names: Elvax, Evatane, Lupolen, Movriton, Ultrathene, Ateva, and Vestypar. These ethylene-vinyl acetate copolymers are available in different grades with respect to the amount of vinyl acetate present in the copolymer, for example, EVA 28 is a copolymer having a vinyl acetate content of 28%.

In one embodiment, at least the skin is made of ethylene-vinyl acetate copolymer. In a further embodiment, the core, the inner compartment, and the skin or the inner compartment and the skin (in a ring without core) are made of ethylene-vinyl acetate copolymers, which copolymers can each be of the same or different grades.

In another embodiment, the inner compartments are made of the same grade of ethylene-vinyl acetate copolymer. However, by electing different polymer grades for the inner compartment, fine-tuning of the flexibility of the ring is possible. The thickness of the skin and the vinyl acetate content of the skin influence the release rate of the active ingredient. The thinner the skin and the higher the vinyl acetate content of the skin, the higher the release rate of the active ingredient.

In one embodiment, EVA copolymers having a vinyl acetate content of from 6% to 40% are used. In another embodiment, EVA copolymers having a vinyl acetate content of from 6% to 33% are used. In a further embodiment, EVA copolymers having a vinyl acetate content of from 9% to 33% are used. In yet another embodiment, EVA copolymers having a vinyl acetate content of from 12% to 33% are used. In another embodiment, the skin is made of EVA copolymers having a vinyl acetate content of from 6% to 33%. In yet another embodiment, the skin is made of EVA copolymers having a vinyl acetate content of from 9% to 33%, for example, EVA 9, EVA 15, EVA 18, EVA 28 or EVA 33. It is known in the art that a lower vinyl acetate content of the EVA copolymers results in a higher the stiffness of the vaginal ring. Moreover, a larger cross-sectional diameter will also result in a higher stifthess, i.e. less flexibility.

A vaginal ring of the present invention can be manufactured by the known process of extrusion, such as co-extrusion and blend extrusion. To obtain the material for the inner compartment comprising the drug, risperidone is mixed with an EVA copolymer. The major step in the mixing process is blend extrusion. Subsequently, the drug/EVA copolymer mixture is co-extruded with the core and skin materials into a three-layered (core comprising) fibre. Alternatively, the drug/EVA copolymer mixture is co-extruded with the skin material into a two-layered fibre (ring without core). After this step, the drug will partly be dissolved in the EVA copolymer. The solubility of the drug in the copolymer is determined by the vinyl acetate content of the EVA copolymer used. Any drug material that is not dissolved will be present as a solid phase in the inner compartment. The solid phase will be in equilibrium with the dissolved phase of the drug, such providing a constant concentration of dissolved active substance close to the rate controlling skin layer. The three-layered or two-layered fibre thus obtained is cut into pieces of a desired length and each piece is assembled to a ring-shaped device in any suitable manner known to the person skilled in this art. The rings are then packed, for example in a suitable sachet, optionally after being sterilized or disinfected.

A person skilled in the art of extrusion will have no difficulty in finding the optimal processing conditions, such as determining the extrusion temperature, extrusion speed, and air gap, for making a three-layered or two-layered fibre containing drug on the basis of methods and procedures known in the art and the description and examples given in this application. A suitable temperature for blend extrusion of the drug/EVA copolymer mixture lies in the range of from 80° C. to 130° C., e.g. approx. 90° C. Suitable temperatures for co-extrusion of the three-layered or two-layered fibre lie in the range of from 80° C. to 150° C.

A preferred temperature for extrusion of risperidone/EVA copolymer mixtures is below the melting point of the drug, i.e. below approximately 170° C. Melting the drug during extrusion may lead to phenomena like delayed crystallization of the drug. In the manufacture of the extended release formulation according to the invention crystalline risperidone is preferred.

In this way, vaginal rings with constant release rates of drug, for example releasing in the range of 0.5 to 10 mg/day of risperidone, can be manufactured.

The vaginal ring according to the present invention can be manufactured in any practical size. In one embodiment, the ring has an outer diameter of between about 50 and 60 mm and in another embodiment between about 52 and 56 mm. In a further embodiment, the cross-sectional diameter is between about 2.0 and 6.0 mm, in a still further embodiment between about 2.5 and 5.0 mm, in another embodiment between about 3.0 and 4.0 mm, and in yet another embodiment it is about 4.0 mm. In one embodiment, the amount of drug contained in the inner compartment is from 5 to 80 wt %, in another embodiment from 10 to 70 wt %, in still another embodiment from 30 to 70 wt %, and in a further embodiment from 40-65 wt %.

In another embodiment, the skin is made of EVA copolymers having a vinyl acetate content of from 9% to 33% and the amount of drug contained in the medicated inner compartment is 40-65 wt %. In yet another embodiment, the skin is made of EVA copolymers having a vinyl acetate content of from 15% to 33%, a thickness in the range of 30 to 200 μm, the copolymer of the inner compartment contains 28 to 33 wt % of vinylacetate and the amount of drug contained in the medicated inner compartment is 30-65 wt %.

In one embodiment the drug delivery system according to the invention is a cylindrical fibre, consisting of a cylindrical inner compartment and a skin covering this compartment. In a particular embodiment the cross-sectional diameter of such a cylindrical fibre is between about 2.5 and 6 mm, in a specific embodiment between about 3.0 and 5.5 mm, and in another embodiment between about 3.5 and 4.5 mm and in yet another embodiment is 4.0 or 5.0 mm. In one embodiment, the surface of the fibre is more than 800 mm², and in another embodiment more than 1000 mm² and in a further embodiment in the order of 1700-2200 mm². Significantly larger surfaces are possible, provided that the design (physical dimensions) of a drug delivery system intended for vaginal use prevents inconvenience for the subject.

In one embodiment said skin has a thickness in the range of 20 to 200 μm, in another 20 to 100 μm. In a still further embodiment said skin has a thickness in the range of 20 to 70 μm. In a still even further embodiment the copolymer of the inner compartment contains 18 to 33 wt % of vinylacetate. In an even further embodiment the copolymer of the inner compartment contains 28 to 33 wt % of vinylacetate. In an even further embodiment the copolymer of the inner compartment comprises 33 wt % of vinylacetate.

The subject invention provides a method of manufacturing the three-layered drug delivery system of the subject invention with risperidone in the intermediate layer, comprising:
(i) producing a medicated homogenous polymer intermediate layer granulate;
(ii) co-extruding a polymer core granulate and the intermediate layer granulate with a polymer skin granulate to form the three-layered drug delivery system.
(iii) collecting the fibre on a reel and forming the extended release formulation according to the invention

The production of the medicated homogeneous polymer intermediate layer granulate comprises:
a. grounding the polymer;
b. dry powder mixing the grounded polymer with risperidone to be loaded in the intermediate layer;
c. blend extruding the resulting powder mixture;
d. cutting the resulting medicated polymer strands into granules, thereby obtaining an intermediate layer granulate;
e. lubricating the intermediate granulate with a lubricant.

### REFERENCES

A. Kydonieus, Marcel Dekker Inc. New York, 1992. Typical diffusion coefficient for steroids, pp. 66-67.
T. S. Harrison and K. L. Goa, Long-acting risperidone: review of its use in schizophrenia.
CNS Drugs (2004), 18:113-132.
A. D. Woolfson, et al, Journal of Controlled Release (2003), 19: 465-476.
J. A. H. van Laarhoven, et al. International Journal of Pharmaceutics (2002), 232: 165.

### FIGURE LEGENDS

FIG. 1 shows a cross-sectional presentation of a three-layered drug (core comprising) delivery system in accordance with the present invention.

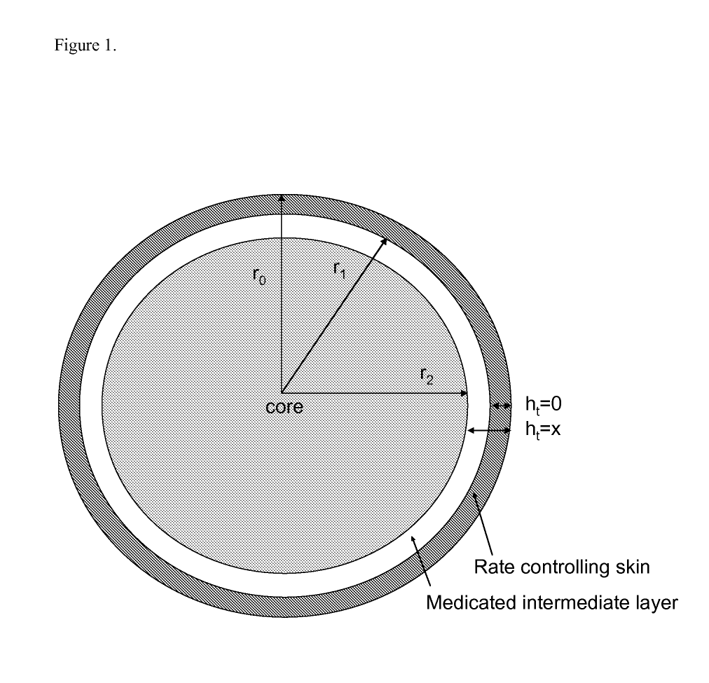

FIG. 2 shows the in vitro release rate of vaginal rings containing risperidone with varying vinyl acetate content of the skin material (Batches 3, 9 and 10). The number in brackets refers to the wt % vinyl acetate of the copolymer.

FIG. 3 shows the in vitro release rate of vaginal rings containing risperidone with a skin thickness of 50 μm (Batch 3) and 200 μm (Batch 1) and EVA 28 as skin material.

FIG. 4 shows the in vitro release of vaginal rings containing 40% (Batch 7) and 60% (Batch 3) of risperidone in the intermediate layer.

The present invention is illustrated by the following Examples.

### EXAMPLE 1

### Preparation of Three-Layered Vaginal Rings Containing Risperidone

Preparation of Three-Layered Vaginal Rings Consisted of Several Steps. First of all, an Inner compartment granulate containing risperidone and EVA 33 copolymer was manufactured in a conventional way by pre-mixing, blend extrusion and lubrication with magnesium stearate. Secondly, a core material of EVA 28 was prepared by lubricating the as—supplied material. Subsequently, the inner compartment granulate, the core granulate and the skin material were co-extruded into a three-layered fibre. The fibre was cut to fibres of a specific length, as described below, after which the fibre ends were welded to a ring.

The inner compartment material was prepared by adding the desired amount of ingredients to a stainless steel drum after which the powder mixture was pre-mixed by rotating the drum on a Rhõnrad at 47 rpm for 60 minutes. The powder mixture was subsequently fed to a Berstorff ZE25 co-rotating twin screw extruder and blend extruded at an extrusion temperature of 80° C. Blend extrusion resulted in strands in which risperidone was homogeneously dispersed in the EVA 33 copolymer. The strands were subsequently granulated to inner compartment granulate. Prior to co-extrusion, the intermediate layer granulate was lubricated with 0.1 wt % magnesium stearate and homogenized in a stainless steel drum on a Rhonrad (barrel-hoop principle) with a fixed rotation speed of 47 rpm for 60 minutes.

The core granulate EVA 28 was also lubricated with 0.1 wt % magnesium stearate and homogenized in stainless steel drum on a Rhonrad (barrel-hoop principle) with a fixed rotation speed of 47 rpm for 60 minutes.

The co-extrusion set-up consisted of a 15 mm skin extruder that processed the skin material, a 18 mm core extruder that processed the core material and an 18 mm inner compartment extruder that processed the inner compartment granulate as delivered by the blend extruder. The melt flows were combined in a spinneret resulting in a three-layered skin-inner compartment-core fibre. The volume flow rate of all three melt flows was controlled by a set of separate spinning pumps. An extrusion temperature of approx. 90° C. was used. Extrusion resulted in a three-layered fibre with a diameter value of approx. 4 mm. The fibre was cooled down to room temperature in a water bath and wound on a reel. The fibre was cut into 157 mm fibres and subsequently the fibres were welded into a ring at 110° C.

TABLE 1
Dimensions of the three-layered risperidone
rings produced comprising an EVA 28 core

| | | | Inner | |
|---|---|---|---|---|
| | | | compartment | Concen- |
| | Skin | Skin thickness | layer thickness | tration drug |
| Batch | material | (μm) | (μm) | (wt %) |
| 1 | EVA 28 | 200 | 200 | 60 |
| 2 | EVA 28 | 200 | 400 | 60 |
| 3 | EVA 28 | 50 | 200 | 60 |
| 4 | EVA 28 | 50 | 400 | 60 |
| 5 | EVA 28 | 200 | 200 | 40 |
| 6 | EVA 28 | 200 | 400 | 40 |
| 7 | EVA 28 | 50 | 200 | 40 |
| 8 | EVA 28 | 50 | 400 | 40 |
| 9 | EVA 33 | 50 | 200 | 60 |
| 10 | EVA 15 | 50 | 200 | 60 |
| 11 | EVA 33 | 200 | 200 | 60 |
| 12 | EVA 15 | 200 | 200 | 60 |

Three-layered rings containing various materials and thicknesses for skin and inner compartment were manufactured (see Table 1).

### In Vitro Release of Three-Layered Vaginal Rings Containing Risperidone

The in vitro release of three-layered vaginal rings containing risperidone was measured in water (buffered at pH 4.4) at 37° C. for at least 24 days. The dimensions of the risperidone rings produced comprising a core are reflected in Table 1.

TABLE 2
Average in vitro release rates (AVG1 and AVG2) from three-
layered vaginal rings containing risperidone (n = 3)

| | AVG1: day 2-12 | AVG2: day 14-24 |
|---|---|---|
| Batch | (mg/day) | (mg/day) |
| 1 | 0.45 | 0.50 |
| 2 | 0.46 | 0.50 |
| 3 | 2.19 | 2.45 |
| 4 | 2.30 | 2.53 |
| 5 | 0.43 | 0.47 |
| 6 | 0.44 | 0.44 |
| 7 | 1.79 | 1.69 |
| 8 | 1.80 | 1.70 |
| 9 | 3.69 | 3.64 |
| 10 | 0.47 | 0.44 |
| 11 | 0.85 | 0.80 |

The average release rates for these batches are given in Table 2. The release rate of the three-layered vaginal rings can be tuned by choosing drug concentration, skin thickness and material.

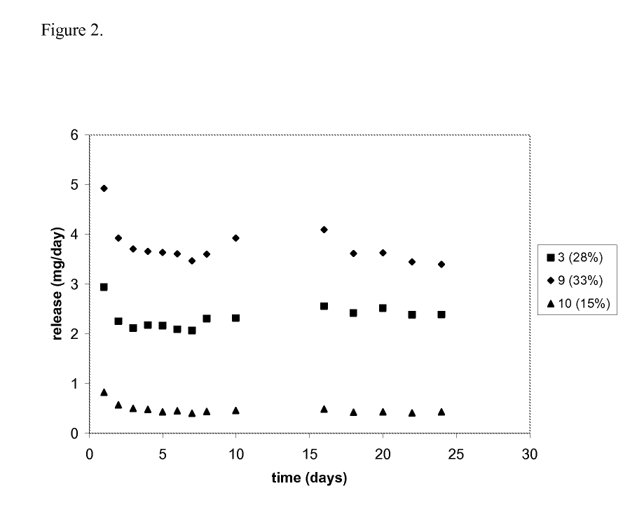

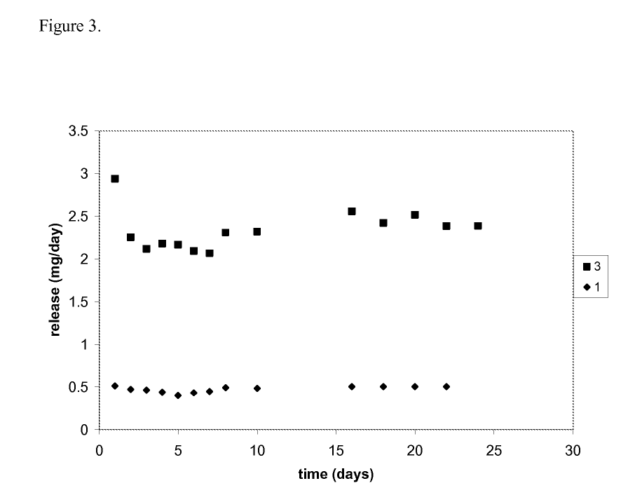

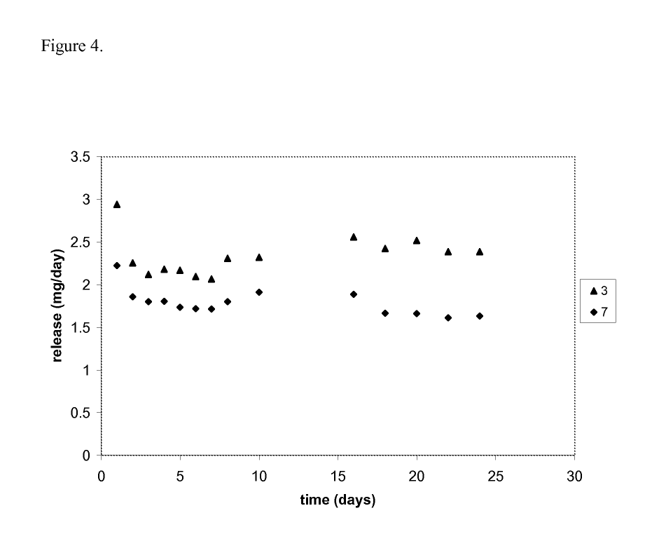

The influence of the vinyl acetate content of the skin material is shown in FIG. 2. The release rate is also influenced by the skin thickness of the vaginal ring as is shown in FIG. 3. FIG. 4 shows the influence of the drug concentration in the inner 15 compartment layer.

The burst-factor and constant release factor of risperidone from three-layered vaginal rings is presented in Table 3. The burst-factor is within the range of −0.5 to 0.5. The constant release factor of risperidone from the three-layered rings is in the range of 0.85 to 1.15.

TABLE 3
Burst-factor and constant release factor of
risperidone from three-layered vaginal rings.

| | | | | | | Constant |
|---|---|---|---|---|---|---|
| | | | | | Burst- | release |
| | Release | Release | AVG1 | AVG2 | factor | factor |
| | DAY 1 | DAY 2 | (mg/ | (mg/ | (D 1 − | AVG1/ |
| Batch | (mg/day) | (mg/day) | day) | day) | D 2)/D 2 | AVG2 |
| 1 | 0.51 | 0.47 | 0.45 | 0.50 | 0.09 | 0.90 |
| 2 | 0.52 | 0.46 | 0.46 | 0.50 | 0.14 | 0.91 |
| 3 | 2.94 | 2.25 | 2.19 | 2.45 | 0.30 | 0.89 |
| 4 | 3.02 | 2.41 | 2.30 | 2.53 | 0.25 | 0.91 |
| 5 | 0.56 | 0.45 | 0.43 | 0.47 | 0.25 | 0.92 |
| 6 | 0.57 | 0.44 | 0.44 | 0.44 | 0.29 | 0.99 |
| 7 | 2.22 | 1.86 | 1.79 | 1.69 | 0.20 | 1.06 |
| 8 | 2.24 | 1.89 | 1.80 | 1.70 | 0.19 | 1.06 |
| 9 | 4.92 | 3.92 | 3.69 | 3.63 | 0.25 | 1.02 |
| 10 | 0.82 | 0.57 | 0.47 | 0.44 | 0.45 | 1.07 |
| 11 | 0.75 | 0.90 | 0.85 | 0.80 | −0.17 | 1.06 |

### EXAMPLE 2

### Test of the Risk of Dose-Dumping

In an in vitro release study in water (buffered at pH 4.4) at 37° C. the risperidone release rate of a vaginal ring according to the invention is compared with a ring, cut into a rod with two open “ring-ends”. The release rate was not significantly affected, indicating that no dose-dumping occurred. Apparently the design of the device according to the invention inherently protects against dose-dumping problems of high-dose drug delivery systems comprising drugs like risperidone.

1-10. (canceled)

11. An extended release formulation comprising solid risperidone, the formulation being in the form of a vaginal device comprising:
(a) an inner compartment made of a thermoplastic polymer, wherein the thermoplastic polymer contains the solid risperidone; and
(b) a skin.

12. The formulation of claim 11, wherein the polymer contains risperidone in the range of 5 to 80 wt %.

13. The formulation of claim 12, wherein the polymer contains risperidone in the range of 30 to 70 wt %.

14. The formulation of claim 11, wherein the skin is substantially continuous.

15. The formulation of claim 11, wherein the device is a ring.

16. The formulation of claim 11, wherein the thermoplastic polymer of the inner compartment is ethylene-vinyl acetate copolymer.

17. The formulation of claim 11, wherein the inner compartment comprises a core, which does not contain solid risperidone.

18. The formulation of claim 11, wherein the skin is made of ethylene-vinyl acetate copolymer.

19. The formulation of claim 16, wherein the ethylene-vinyl acetate copolymer has a vinyl acetate content in the range of 6 to 40%.

20. The formulation of claim 18, wherein the ethylene-vinyl acetate copolymer has a vinyl acetate content in the range of 6 to 40%.

21. The formulation of claim 1, wherein the device is obtainable by extrusion or by co-extrusion.

22. A method of treating bipolar mania in a patient in need of treatment, the method comprising administering to the patient an extended release formulation comprising solid risperidone, wherein the formulation is in the form of a vaginal device comprising:
(a) an inner compartment made of a thermoplastic polymer, wherein the thermoplastic polymer contains the solid risperidone; and
(b) a skin, wherein the release rate of risperidone is about 0.5 to 10 mg/day.

23. A method of treating schizophrenia in a patient in need of treatment, the method comprising administering to the patient an extended release formulation comprising solid risperidone, wherein the formulation is in the form of a vaginal device comprising:
(a) an inner compartment made of a thermoplastic polymer, wherein the thermoplastic polymer contains the solid risperidone; and
(b) a skin, wherein the release rate of risperidone is about 0.5 to 10 mg/day.
